(19) United States
(12) Patent Application Publication
(10) Pub. No.: US 2026/0268136 A1
(43) Pub. Date: Sep. 10, 2026

(54) APPARATUS AND METHOD CONFIGURED TO GENERATE BIOSIGNAL DATA USING A DIFFUSION MODEL

(71) Applicant: Electronics and Telecommunications Research Institute, Daejeon (KR)

(72) Inventors: Hyeonjeong LEE, Daejeon (KR); Minseong KIM, Daegu (KR)

(73) Assignee: Electronics and Telecommunications Research Institute, Daejeon (KR)

(21) Appl. No.: 19/558,920

(22) Filed: Mar. 6, 2026

(30) Foreign Application Priority Data
Mar. 6, 2025 (KR) 10-2025-0028867

(51) Int. Cl. G06N 3/08; G06N 3/0455; G06N 3/0464

(52) U.S. Cl. CPC G06N 3/08; G06N 3/0455; G06N 3/0464

(57) ABSTRACT

An apparatus configured to generate biosignal data using a diffusion model comprises: a biosignal data input module configured to receive reference biosignal data and to collect data samples corresponding to a predetermined biosignal condition; a processor configured to generate biosignal data reflecting a predetermined health state by utilizing the collected data samples and a diffusion model; and a biosignal data output module configured to store the generated biosignal data and to output the generated biosignal data for use in training a medical artificial intelligence (AI) model.

### CROSS-REFERENCE TO RELATED APPLICATION

This application claims priority under 35 U.S.C. § 119 to Korean Patent Application No. 10-2025-0028867, filed on Mar. 6, 2025, in the Korean Intellectual Property Office, the disclosure of which is incorporated herein in its entirety by reference.

### BACKGROUND

The present invention relates to an apparatus and method configured to generate biosignal data using a diffusion model.

Biosignal data utilized for diagnosing and monitoring an individual's health status includes various physiological indicators, such as heart rate, blood pressure, skin temperature, and respiration rate. Such data plays a crucial role in non-invasive healthcare, remote monitoring, and personalized medical services, and its importance is increasing with advancements of precision and preventive medicine technologies.

However, biosignal data exhibits characteristics that vary among individual and is prone to noise due to the influence of various factors such as the external environment, physical condition, sensor contact status, and movement. Furthermore, it is difficult to obtain sufficient data for predetermined diseases or conditions (e.g., hypotension or hypertension), which may negatively affect the learning and generalization performance of artificial intelligence (AI) models that utilize it.

In particular, biosignal data corresponding to predetermined conditions, such as hypotension, hypertension, and cardiac arrhythmia, is extremely scarce compared to data corresponding to normal states, thereby causing a problem where AI-based prediction models have difficulty sufficiently learning these predetermined states.

Accordingly, there is a need for a technology to solve the problem of scarcity and imbalance of biosignal data and effectively generate biosignal data corresponding to predetermined conditions (e.g., blood pressure levels).

### SUMMARY

A technical problem to be solved by the present disclosure is to provide an apparatus and method configured to generate biosignal data using a diffusion model, capable of solving the problem of biosignal data scarcity and overcome the limitations of data collection for predetermined biosignal conditions.

Furthermore, an embodiment provides an apparatus and method configured to generate biosignal data using a diffusion model, capable of mitigating the data imbalance problem of reference biosignal data and enable data generation that considers various patient groups.

Furthermore, an embodiment provides an apparatus and method configured to generate biosignal data using a diffusion model, capable of improving accuracy when training a medical AI model and enable precise prediction corresponding to predetermined health states.

According to an aspect, an embodiment provides an apparatus configured to generate biosignal data using a diffusion model, comprising: a biosignal data input module configured to receive reference biosignal data and to collect data samples corresponding to a predetermined biosignal condition; a processor configured to generate biosignal data reflecting a predetermined health state by utilizing the collected data samples and a diffusion model; and a biosignal data output module configured to store the generated biosignal data and to transmit the generated biosignal data to an external device through a communication interface, for use in training a medical artificial intelligence (AI) model.

The processor may guide data generation by considering a predetermined biosignal group condition, including a hypotension group and a hypertension group, during biosignal data generation.

The processor may generate biosignal data by reflecting a change in at least one of a time-varying blood pressure value and a time-varying heart rate value, during biosignal data generation.

The processor may learn to transform a structure of the reference biosignal data by adding probabilistic noise to the reference biosignal data in a forward process of the diffusion model.

The processor may learn to restore the reference biosignal data by gradually removing noise in a reverse process of the diffusion model.

The processor may evaluate similarity between the generated biosignal data and the reference biosignal data, and perform a data distribution comparison and a signal waveform comparison.

The processor may calculate a mean and a variance by performing Principal Component Analysis (PCA), and perform a biosignal data post-processing function by removeing outliers having a variance of x times (where x is a natural number) or more from the mean.

When performing the biosignal data post-processing function, to enhance the quality of the data generated via the diffusion model, the processor may apply a Recursive Exponential Averaging technique to reduce signal noise and maintain data continuity.

When performing the biosignal data post-processing function, the processor may detect an outlier and remove data with low reliability by comparing reference biosignal data with generated biosignal data through a filtering function.

The processor may learn patterns of biosignal data through a diffusion model using a CNN-based encoder-decoder structure, and perform data transformation in the forward and reverse processes of the diffusion model.

According to another aspect, an embodiment provides a method configured to generate biosignal data using a diffusion model, comprising: receiving, by a processor through a biosignal data input module, reference biosignal data; collecting data samples corresponding to a predetermined biosignal condition; generating, by the processor, biosignal data reflecting a predetermined health state by utilizing the reference biosignal data samples collected through the biosignal data input module and a diffusion model; storing, by the processor through a biosignal data output module, the generated biosignal data; and transmitting the generated biosignal data to an external device through a communication interface, for use in training a medical artificial intelligence (AI) model.

The generating of the biosignal data may include guiding data generation by considering a predetermined biosignal group condition, containing a hypotension group and a hypertension group, during the biosignal data generation.

The generating of the biosignal data may include generating biosignal data by reflecting a change in at least one of a time-varying blood pressure value and a time-varying heart rate value, during the biosignal data generation.

The generating of the biosignal data may include learning to transform the structure of the reference biosignal data by adding probabilistic noise to the reference biosignal data in a forward process of the diffusion model.

The generating of the biosignal data may further include learning to restore the reference biosignal data by gradually removing noise in a reverse process of the diffusion model.

The generating of the biosignal data may include evaluating similarity between the generated biosignal data and the reference biosignal data, and performing a data distribution comparison and a signal waveform comparison.

The generating of the biosignal data may include calculates a mean and a variance by performing Principal Component Analysis (PCA), and performing a biosignal data post-processing function by removeing outliers having a variance of x times (where x is a natural number) or more from the mean.

The generating of the biosignal data, when performing the biosignal data post-processing function, may include applying a Recursive Exponential Averaging technique to reduce signal noise and maintain data continuity.

The generating of the biosignal data, when performing the biosignal data post-processing function may include detecting an outlier by comparing reference biosignal data with generated biosignal data through a filtering function, and removing data with low reliability.

The generating of the biosignal data may include learning patterns of biosignal data through a diffusion model using a CNN-based encoder-decoder structure, and performing data transformation in the forward and reverse processes of the diffusion model.

According to above aspects, an embodiment may solve the problem of reference biosignal data scarcity and enhance the quality of training data for medical AI models by generating biosignal data that meets predetermined biosignal conditions (e.g., blood pressure, heart rate, etc.) using a diffusion model.

Furthermore, according to above aspects, an embodiment may provide more realistic biosignal data compared to conventional data augmentation techniques by applying a diffusion model that performs forward noise addition and reverse restoration to generate high-quality biosignal data similar to actual biosignal data.

Furthermore, according to above aspects, an embodiment may improve the prediction performance of a medical artificial intelligence (AI) model trained using the biosignal data by applying post-processing techniques such as outlier removal and Principal Component Analysis (PCA) to evaluate the quality of the generated biosignal data and increase its reliability.

Furthermore, according to above aspects, an embodiment may enhance the precision of AI-based diagnosis and prediction systems that reflect various health states by enabling the generation of customized biosignal data according to a patient's predetermined biosignal state (e.g., hypertension, hypotension), and learning from this biosignal data.

Furthermore, according to above aspects, an embodiment may increase the efficiency of medical data acquisition by reducing the cost and time required to directly collect data from actual patients and enabling the generated data for use in medical research and AI model development.

### BRIEF DESCRIPTION OF THE DRAWINGS

FIG. 1 is an exemplary diagram showing a schematic configuration of an apparatus configured to generate biosignal data using a diffusion model according to the aspect of the present invention.

FIG. 2 is a flowchart for describing a method configured to generate biosignal data using a diffusion model according to the another aspect of the present invention.

FIG. 3 is a flowchart for describing a process of generating biosignal data based on a Diffusion Model and training a blood pressure (SBP, DBP) estimation model by utilizing the generated data according to an embodiment of the present invention.

### DETAILED DESCRIPTION

Hereinafter, embodiments of an apparatus and method configured to generate biosignal data using a diffusion model according to an embodiment of the present invention will be described.

In this process, the thickness of lines or the size of components shown in the drawings may be exaggerated for clarity and convenience of description. In addition, the terms used below are defined in consideration of their functions in the present disclosure and may vary according to the intention or custom of a user or operator. Therefore, the definition of these terms should be made based on the content throughout this specification.

Hereinafter, embodiments of the present invention will be described in detail with reference to the accompanying drawings so that those of ordinary skill in the art to which the present invention pertains may easily carry out the present invention. However, the present invention may be implemented in various different forms and is not limited to the embodiments described herein. In the drawings, parts unrelated to the description have been omitted to clearly describe the present invention, and similar reference numerals have been used for similar parts throughout the specification.

Throughout the specification, when a part "includes" a component, unless explicitly stated otherwise, it means that the part does not exclude other components but may include other components.

The implementations described herein may be implemented, for example, as a method or process, an apparatus, a software program, a data stream, or a signal. Even if discussed only in the context of a single form of implementation (e.g., discussed only as a method), the implementation of the features discussed may also be implemented in other forms (e.g., an apparatus or a program). An apparatus may be implemented with appropriate hardware, software, and firmware, etc. A method may be implemented in an apparatus such as a processor, which generally refers to a processing device including, for example, a computer, a microprocessor, an integrated circuit, or a programmable logic device, etc.

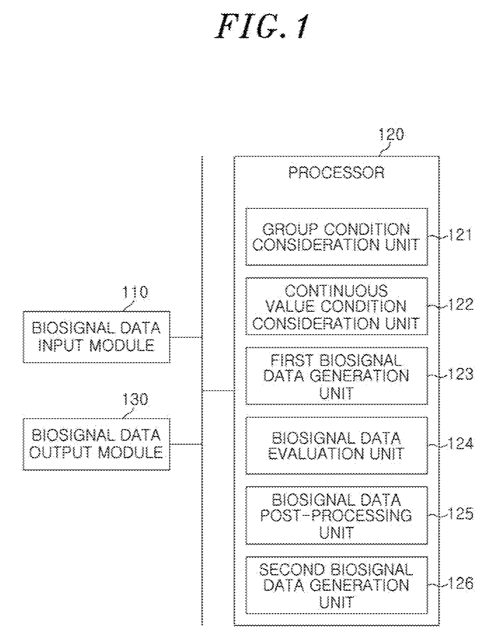

FIG. 1 is an exemplary diagram showing a schematic configuration of an apparatus configured to generate biosignal data using a diffusion model according to the aspect of the present invention.

Referring to FIG. 1, an apparatus configured to generate biosignal data using a diffusion model according to the aspect may include a biosignal data input module 110, a processor 120, and a biosignal data output module 130.

The biosignal data input module 110 performs a role of receiving reference biosignal data and collecting data samples that meet predetermined biosignal conditions (e.g., blood pressure, heart rate, etc.). In an embodiment, the biosignal data input module 110 may include at least one biosignal sensor configured to measure physiological signals, such as an electrocardiogram (ECG) sensor, a photoplethysmography (PPG) sensor, and a blood pressure (BP) sensor.

In an exemplary embodiment, the biosignal data input module 110 collects biosignal data such as an electrocardiogram (ECG), a photoplethysmogram (PPG), and blood pressure (BP), and selects N samples (N-shot data) to be transferred to the processor 120 for training the diffusion model. The selected data at this time may include data corresponding to a predetermined health state (e.g., blood pressure, etc.) and is utilized as important reference data during diffusion model training.

The processor 120 performs a role of generating biosignal data by utilizing a diffusion model and evaluating and improving its quality. That is, the processor 120 generates biosignal data by utilizing the diffusion model and allows for the provision of high-quality data reflecting a predetermined health state.

At this time, in this embodiment, the diffusion model is a probabilistic data generation model that includes a forward process and a reverse process.

More specifically, the diffusion model may operate in a manner that it transforms the original shape of the data by gradually adding noise to the input biosignal data in the forward process, and then restores biosignal data similar to the original data while gradually removing the noise in the reverse process. That is, in the forward process of the diffusion model, a transformation is performed to add probabilistic noise to the reference biosignal data, and in the reverse process, learning may proceed to restore the reference biosignal data by gradually removing noise from the noised data.

At this time, in the forward process, to reflect predetermined conditions, a group condition (e.g., it may reflect a predetermined physiological state such as a hypotension group, a hypertension group, etc.) and a continuous value condition (e.g., it may reflect continuous biosignal characteristics such as a blood pressure value of 60, a heart rate of 80, etc.) may be applied as a guide.

Furthermore, the reverse process of the diffusion model is based on a probabilistic model and may be guided to reflect a predetermined health state. When the diffusion model is sufficiently trained, the diffusion model starts from data in a complete noise state and gradually transforms it into a form similar to the reference biosignal data.

Here, as an artificial neural network for implementing the diffusion model, a CNN (Convolutional Neural Network), an RNN (Recurrent Neural Network), an LSTM (Long Short-Term Memory), or the like may be utilized.

In an exemplary embodiment, when utilizing a CNN as the artificial neural network for implementing the diffusion model, it is advantageous for analyzing the morphological patterns of biosignal data. When utilizing an RNN or LSTM, it is advantageous for reflecting the time-series characteristics of biosignal data.

In this embodiment, a CNN-based encoder-decoder structure may be used to learn the patterns of biosignal data in the forward and reverse processes.

Furthermore, in this embodiment, the quality of the biosignal data generated through the diffusion model may be evaluated, and if necessary, post-processing techniques may be applied to enhance the reliability of the data, and data with low quality or a large difference from actual biosignal data (i.e., an outlier) may be removed.

In this embodiment, the data quality may be improved by calculating the mean and variance of the generated data using PCA (Principal Component Analysis), and then removing data that deviates from the mean by x times the variance or more.

In this embodiment, the similarity may be evaluated by comparing the statistical distribution of the reference biosignal data with the distribution of the generated data.

As such, by applying the diffusion model in this embodiment, reliable data may be generated even for predetermined health states (e.g., hypotension, hypertension) where reference biosignal data is scarce. Furthermore, by applying the diffusion model in this embodiment, the generated data may be combined with existing N-shot data and utilized as training data for a medical AI model, and the prediction performance may be enhanced. Furthermore, by providing a group condition and a continuous value condition as a guide in this embodiment, data reflecting a predetermined biosignal condition may be generated. Furthermore, through the application of the forward and reverse processes of the diffusion model in this embodiment, more realistic biosignal data may be generated than with conventional methods. Furthermore, reliable data may be obtained through post-processing steps such as outlier removal and PCA in this embodiment.

The processor 120 may include, as detailed components, a group condition consideration unit 121, a continuous value condition consideration unit 122, a first biosignal data generation unit 123, a biosignal data evaluation unit 124, a biosignal data post-processing unit 125, and a second biosignal data generation unit 126. These detailed components may be implemented in the form of logic that performs each function, and the actual operation may be performed by the processor 120.

At least one of the modules or units (collectively "components" in this paragraph) represented by a block or an equivalent indication in FIG. 1 may be implemented or embodied by analog and/or digital circuits including one or more of a logic gate, an integrated circuit, a microprocessor, a microcontroller, a memory circuit, a passive electronic component, an active electronic component, an optical component, and the like. Alternatively or additionally, these components may be implemented or embodied by software including one or more instructions stored in an internal or external storage medium that is readable by at least one processor. For example, the at least one processor may invoke at least one of the one or more instructions stored in the storage medium, and execute it, with or without using one or more other components under the control of the at least one processor. This allows the at least one processor to perform at least one function or operation described above as being performed by each of the components according to the at least one instruction invoked. Here, the at least one processor may include a central processing unit (CPU), a graphic processing unit (GPU), another type of microprocessor, not being limited thereto

The group condition consideration unit 121 may perform a role of guiding data generation by considering a predetermined biosignal group condition (e.g., hypotension group, hypertension group) during the generation of biosignal data.

In an exemplary embodiment, the group condition consideration unit 121 sets a group condition such as a hypotension group or a hypertension group during model training, so that a predetermined health state may be reflected during biosignal data generation. For instance, it may be set to generate biosignal data corresponding to the hypotension group.

The continuous value condition consideration unit 122 may consider the change of a continuous value when generating biosignal data by reflecting a predetermined continuous value (e.g., a blood pressure value of 60). That is, the continuous value condition consideration unit 122 guides data generation by considering a continuous value condition such as a blood pressure value or a heart rate.

In an exemplary embodiment, when the continuous value condition consideration unit 122 sets a blood pressure value of 60 as the continuous value condition, it generates PPG data that belongs to the hypotension group and has a blood pressure value of 60.

The first biosignal data generation unit 123 may generate initial biosignal data using the diffusion model. That is, the first biosignal data generation unit 123 generates initial biosignal data by applying the forward and reverse processes of the diffusion model.

Here, the forward process is a process of transforming the shape of the data by gradually adding noise to the actual biosignal data, and the reverse process learns a method of restoring the reference biosignal data from the data to which noise has been added, and adjusts the generated data to be similar to the actual data.

The biosignal data evaluation unit 124 may perform a role of evaluating the quality of the generated biosignal data and verifying its reliability by comparing the similarity with existing data.

For reference, similarity evaluation methods include a data distribution comparison (i.e., evaluating by analyzing the difference between the distribution of reference biosignal data and the distribution of the refined generated data), and a waveform comparison (i.e., evaluating the quality by comparing the morphological similarity between the existing data and the generated data).

The biosignal data post-processing unit 125 may remove outliers by reflecting the quality evaluation results and refine the data by utilizing techniques such as Principal Component Analysis (PCA). That is, the biosignal data post-processing unit 125 applies post-processing techniques such as outlier removal and Principal Component Analysis (PCA) to further enhance the quality of the generated data.

In an exemplary embodiment, the biosignal data post-processing unit 125 removes outlier data having a variance of x times (where x is a natural number) or more from the mean, after calculating the mean and variance by utilizing PCA.

The second biosignal data generation unit 126 may generate additional biosignal data based on the post-processed data or generate more precise biosignal data by combining it with existing data.

In an exemplary embodiment, the second biosignal data generation unit 126 may construct a dataset for training a more accurate medical AI model by combining N samples (N-shot data) for training the existing diffusion model with the refined data.

The biosignal data output module 130 may store the generated biosignal data and output it for use in training a medical artificial intelligence (AI) model, etc. This supports the medical artificial intelligence (AI) model to learn reliable biosignal data reflecting a predetermined health state. In an embodiment, the biosignal data output module 130 may store the generated biosignal data in a non-transitory storage medium and transmit the generated biosignal data to an external device or system through a wired or wireless communication interface. For example, the generated biosignal data may be transmitted to a training server that executes a medical artificial intelligence (AI) model to update model parameters based on the generated biosignal data. In another example, the generated biosignal data may be provided to a patient monitoring device, an electronic medical record (EMR) system, or a clinical decision support system (CDSS) to (i) retrain or fine-tune a disease prediction model, (ii) validate model performance under a specific physiological condition, or (iii) generate a health risk assessment result for a subject.

In some embodiments, the output biosignal data may be converted into a standardized medical data format and stored in a hospital database, and the medical AI model may generate a diagnostic score, anomaly detection result, or risk stratification output based on the stored biosignal data.

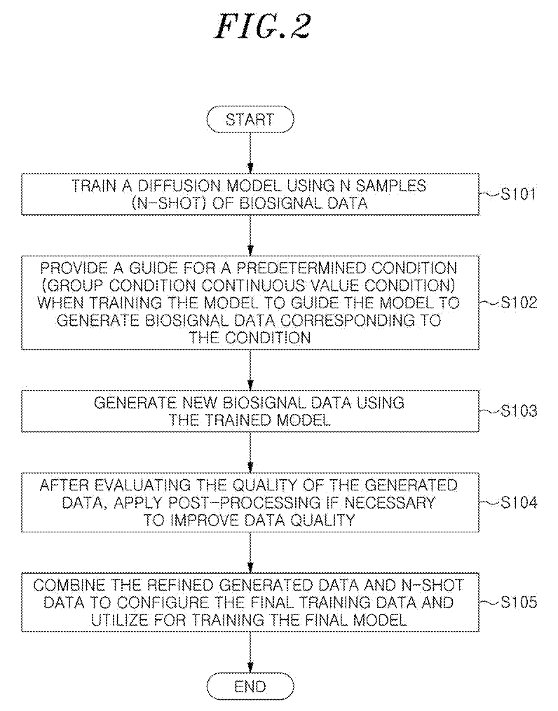

FIG. 2 is a flowchart for describing a method configured to generate biosignal data using a diffusion model according to another aspect of the present invention.

Referring to FIG. 2, the processor 120 performs diffusion model training using biosignal data samples (S101).

In an exemplary embodiment, the biosignal data input module 110 selects N samples (N-shot data) and transfers the N samples to the processor 120. The processor 120 trains the diffusion model based on N samples (N-shot data) from the reference biosignal data.

At this time, the diffusion model is trained to learn the distribution of actual data through forward noise addition and reverse restoration, and to be able to generate new biosignal data.

The processor 120 provides a guide that considers predetermined conditions (S102).

In an exemplary embodiment, the group condition consideration unit 121 of the processor 120 sets a group condition (e.g., hypotension, hypertension) for the biosignal data to be generated. The continuous value condition consideration unit 122 of the processor 120 guides data generation by reflecting continuous numerical conditions such as a blood pressure value of 60 and a heart rate of 80. This allows the diffusion model to optimize data generation that reflects a predetermined physiological state.

The processor 120 generates new biosignal data using the trained diffusion model (S103).

In an exemplary embodiment, the first biosignal data generation unit 123 of the processor 120 generates new biosignal data by utilizing the trained diffusion model. At this time, the generated biosignal data reflects existing biosignal patterns and may appropriately simulate biosignals according to a predetermined health condition. In this process, the forward process (noise addition) and the reverse process (noise removal) are repeated to generate biosignal data similar to the existing data.

The processor 120 applies quality evaluation and post-processing to the generated biosignal data (S104).

In an exemplary embodiment, the biosignal data evaluation unit 124 of the processor 120 evaluates the quality of the generated data and compares its similarity with existing data. Reflecting the quality evaluation results, the biosignal data post-processing unit 125 removes outliers and enhances reliability by applying techniques such as Principal Component Analysis (PCA).

The processor 120 combines the refined generated data with the existing N-shot data to construct the final training data and uses it for training the final model (S105).

In an exemplary embodiment, the second biosignal data generation unit 126 of the processor 120 constructs the final training data by combining the refined generated data with the existing N-shot data. This optimizes the quality of the training data so that the medical AI model can perform more accurate predictions.

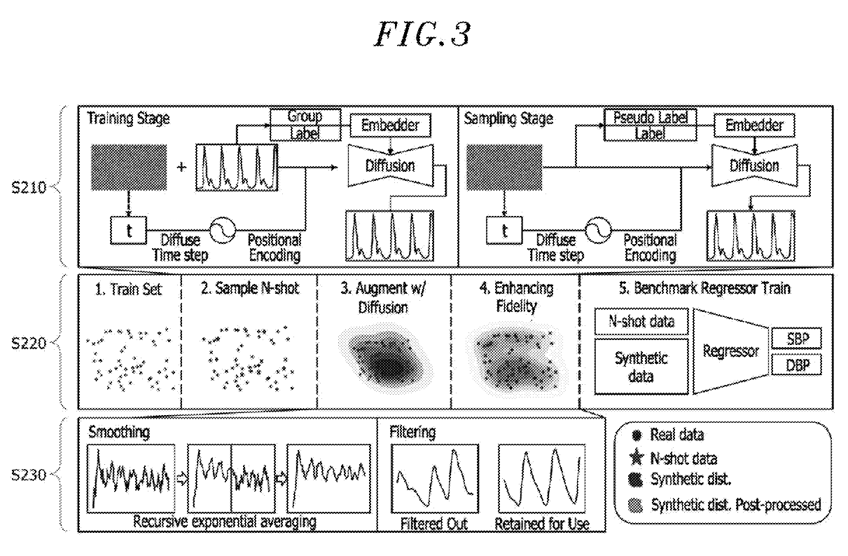

FIG. 3 is a flowchart for describing a process of generating biosignal data based on a Diffusion Model and training a blood pressure (SBP, DBP) estimation model by utilizing the generated data according to an embodiment of the present invention.

This embodiment provides a method for enhancing the performance of a blood pressure estimation model by utilizing a small amount of biosignal data (N-shot data) together with biosignal data generated through a diffusion model.

The configuration of FIG. 3 is largely divided into a diffusion model training stage (S210), a data generation and training data construction stage (S220), and a post-processing stage (S230), and the main processes performed in each stage (S210, S220, S230) are as follows.

The diffusion model training stage (Training Stage & Sampling Stage) (S210) may include a process in which the processor 120 trains the diffusion model (Training Stage) and generates new biosignal data based on the trained model (Sampling Stage).

In this embodiment, the diffusion model may be trained by utilizing a small amount of biosignal data (N-shot data) together with a Group Label, Diffuse Time Step information, and Positional Encoding. Furthermore, when generating new biosignal data using the trained diffusion model, a pseudo-label and a condition (Label) may be added to generate biosignal data reflecting a predetermined health state.

The data generation and training data construction stage (S220) includes a process of building data for training the blood pressure estimation model, and may include the following detailed steps.

As a step of building an actual data training set (Train Set), the biosignal data used in this embodiment may include biosignal data such as an electrocardiogram (ECG), a photoplethysmogram (PPG), and blood pressure (BP), and a training set including existing measured biosignal data (Real Data) may be constructed.

As a step of sampling a small amount of data (Sample N-shot), initial training of the blood pressure estimation model may be performed by sampling some of the entire training data as N-shot data. Here, the N-shot data may be utilized as important reference data in the diffusion model training and sampling process (S210) of this embodiment.

As a step of data augmentation using a diffusion model (Augment with Diffusion), new biosignal data can be generated using the diffusion model, and the dataset may be expanded by combining it with existing N-shot data. The diffusion model of this embodiment can perform the function of generating high-quality data similar to actual biosignal data by reflecting predetermined conditions (e.g., group condition, continuous value condition).

As a step of enhancing the quality of generated data (Enhancing Fidelity), a quality evaluation and post-processing process may be performed to increase the quality of the generated biosignal data. By comparing the distribution of existing data with that of generated data, low-quality data may be filtered while maintaining signals similar to the actual data.

As a step of training the blood pressure estimation model (Benchmark Regressor Train), in this embodiment, N-shot data and data generated through the diffusion model may be used together in the process of training the blood pressure estimation model. The trained blood pressure estimation model may be used to predict Systolic Blood Pressure (SBP) and Diastolic Blood Pressure (DBP), and may enable more precise blood pressure estimation than conventional methods.

In the data post-processing stage (S230), a post-processing process for enhancing the quality of the generated biosignal data may be applied.

The data post-processing stage (S230) may include signal smoothing, and filtering and data selection steps as detailed steps.

In the signal smoothing step, a Recursive Exponential Averaging technique may be applied to the generated biosignal data to reduce signal noise and ensure data continuity.

In the filtering and data selection step, only reliable data may be maintained as training data by performing outlier removal or filtering of low-quality data. The effect of enhancing the learning performance of the blood pressure estimation model may be obtained through filtering.

This embodiment allows for solving the problem of reference biosignal data scarcity and enhancing the quality of training data for medical AI models by generating biosignal data that meets predetermined biosignal conditions (e.g., blood pressure, heart rate, etc.) using a diffusion model.

Furthermore, this embodiment allows for providing more realistic biosignal data compared to conventional data augmentation techniques by applying a diffusion model that performs forward noise addition and reverse restoration to generate high-quality biosignal data similar to actual biosignal data.

Furthermore, this embodiment allows for improving the prediction performance of a medical artificial intelligence (AI) model trained using the biosignal data by applying post-processing techniques such as outlier removal and Principal Component Analysis (PCA) to evaluate the quality of the generated biosignal data and increase its reliability.

Furthermore, this embodiment allows for enhancing the precision of AI-based diagnosis and prediction systems that reflect various health states by enabling the generation of customized biosignal data according to a patient's predetermined biosignal state (e.g., hypertension, hypotension), and learning from this biosignal data.

Furthermore, this embodiment allows for increasing the efficiency of medical data acquisition by reducing the cost and time required to directly collect data from actual patients and enabling the generated data for use in medical research and AI model development.

The present invention has been described above with reference to the embodiments shown in the drawings, but this is merely exemplary, and it will be understood by those of ordinary skill in the art to which the technology pertains that various modifications and equivalent other embodiments are possible therefrom.

Therefore, the technical protection scope of the present invention should be determined by the claims below. In addition, the implementations described in this specification may be implemented, for example, as a method or process, an apparatus, a software program, a data stream, or a signal. Even if discussed only in the context of a single form of implementation (e.g., discussed only as a method), the implementation of the features discussed may also be implemented in other forms (e.g., an apparatus or a program). An apparatus may be implemented with appropriate hardware, software, and firmware, etc. A method may be implemented in an apparatus such as a processor, which generally refers to a processing device including, for example, a computer, a microprocessor, an integrated circuit, or a programmable logic device, etc.

What is claimed is:

1. An apparatus configured to generate biosignal data using a diffusion model, the apparatus comprising:
a biosignal data input module configured to receive reference biosignal data and to collect data samples corresponding to a predetermined biosignal condition;
a processor configured to generate biosignal data reflecting a predetermined health state by utilizing the collected data samples and the diffusion model; and
a biosignal data output module configured to store the generated biosignal data and to transmit the generated biosignal data to an external device through a communication interface, for use in training a medical artificial intelligence (AI) model.

2. The apparatus of claim 1, wherein the processor guides data generation by considering a predetermined biosignal group condition, including a hypotension group and a hypertension group, during biosignal data generation.

3. The apparatus of claim 1, wherein the processor generates the biosignal data by reflecting a change in at least one of a time-varying blood pressure value and a time-varying heart rate value, during the biosignal data generation.

4. The apparatus of claim 1, wherein the processor learns to transform a structure of the reference biosignal data by adding probabilistic noise to the reference biosignal data in a forward process of the diffusion model.

5. The apparatus of claim 4, wherein the processor learns to restore the reference biosignal data by gradually removing noise in a reverse process of the diffusion model.

6. The apparatus of claim 1, wherein the processor evaluates a similarity between the generated biosignal data and the reference biosignal data, and performs a data distribution comparison and a signal waveform comparison.

7. The apparatus of claim 1, wherein the processor calculates a mean and a variance by performing Principal Component Analysis (PCA), and performs a biosignal data post-processing function by removing outliers having a variance of x times (where x is a natural number) or more from the mean.

8. The apparatus of claim 7, wherein, when performing the biosignal data post-processing function, the processor applies a recursive exponential averaging technique to reduce signal noise and maintain data continuity.

9. The apparatus of claim 7, wherein, when performing the biosignal data post-processing function, the processor detects an outlier by comparing the reference biosignal data with the generated biosignal data through a filtering function, and removes data with low reliability.

10. The apparatus of claim 1, wherein the processor learns a pattern of the biosignal data through a diffusion model using a Convolutional Neural Network (CNN)-based encoder-decoder structure, and performs a data transformation in a forward process and a reverse process of the diffusion model.

11. A method configured to generate biosignal data using a diffusion model, the method comprising:
receiving, by a processor via a biosignal data input module, reference biosignal datal;
collecting data samples corresponding to a predetermined biosignal condition;
generating, by the processor, biosignal data reflecting a predetermined health state by utilizing the collected data samples and the diffusion model;
storing, by the processor via a biosignal data output module, the generated biosignal data; and
transmitting the generated biosignal data to an external device through a communication interface, for use in training a medical artificial intelligence (AI) model.

12. The method of claim 11, wherein the generating the biosignal data includes guiding data generation by considering a predetermined biosignal group condition, containing a hypotension group and a hypertension group, during the biosignal data generation.

13. The method of claim 11, wherein the generating the biosignal data includes generating the biosignal data by reflecting a change in at least one of a time-varying blood pressure value and a time-varying heart rate value, during the biosignal data generation.

14. The method of claim 11, wherein the generating the biosignal data includes learning to transform a structure of the reference biosignal data by adding probabilistic noise to the reference biosignal data in a forward process of the diffusion model.

15. The method of claim 14, wherein the generating the biosignal data further includes learning to restore the reference biosignal data by gradually removing noise in a reverse process of the diffusion model.

16. The method of claim 11, wherein the generating the biosignal data includes, to verify the reliability of the generated biosignal data, evaluating a similarity between the generated biosignal data and the reference biosignal data, and performing a data distribution comparison and a signal waveform comparison.

17. The method of claim 11, wherein the generating the biosignal data includes calculateing a mean and a variance by performing Principal Component Analysis (PCA), and performing a biosignal data post-processing function by removing outliers having a variance of x times (where x is a natural number) or more from the mean.

18. The method of claim 17, wherein the performing the biosignal data post-processing function includes applying a recursive exponential averaging technique to reduce signal noise and maintain data continuity.

19. The method of claim 17, wherein the performing the biosignal data post-processing function includesdetecting an outlier by comparing the reference biosignal data with the generated biosignal data through a filtering function, and removing data with low reliability.

20. The method of claim 11, wherein the generating the biosignal data includes learning a pattern of the biosignal data through a diffusion model using a Convolutional Neural Network (CNN)-based encoder-decoder structure, and performing a data transformation in a forward process and a reverse process of the diffusion model.
